SEQUENCE LISTING (6 sequences)

SEQ ID NO: 1 - DNA, 11 bp, Salmonella enterica
aaaagagataa

SEQ ID NO: 2 - DNA, 11 bp, Salmonella enterica
gaaggagtgga

SEQ ID NO: 3 - DNA, 20 bp, Artificial Sequence
  Oligonucleotide for in vitro amplification of rbf M gene
cttgggttacaaattaggca

SEQ ID NO: 4 - DNA, 20 bp, Artificial Sequence
  Oligonucleotide for in vitro amplification of rfb M gene
atcttttacaagaccgcgag

SEQ ID NO: 5 - DNA, 20 bp, Artificial Sequence
  Oligonucleotide for in vitro amplification of rfb K gene
ccccctgaagttaattgaga

SEQ ID NO: 6 - DNA, 20 bp, Artificial Sequence
  Oligonucleotide for in vitro amplification of rfb K gene
ccatttaatcctcaccctct

(19) United States
(12) Patent Application Publication
(10) Pub. No.: US 2003/0082752 A1
(43) Pub. Date: May 1, 2003

(54) Enzymatic process for producing GDP-alpha-D-mannose, a GDP mannose pyrophosphorylase and phosphomannomutase suitable for that process, the extraction of the said enzymes, and an enzyme test

(76) Inventors: Jorg Eberhard Ritter, Niederzier (DE); Lothar Elling, Aachen (DE); Maria-Regina Kula, Niederzier (DE); Stefen Verseck, Wuppertal (DE)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/812,864

(22) Filed: Mar. 19, 2001

Related U.S. Application Data
(62) Division of application No. 08/894,933, filed on Apr. 30, 1998, now abandoned.

(30) Foreign Application Priority Data
Mar. 1, 1996 (DE) PCT/DE96/00371
Mar. 3, 1995 (DE) 195 07 449.1
May 15, 1995 (DE) 195 17 093.8
Feb. 23, 1996 (DE) 196 06 651.4

(51) Int. Cl. C12P 19/30; C12N 9/16

(52) U.S. Cl. 435/89; 435/196

(57) ABSTRACT

The invention provides a GDP-mannose-pyrophosphorylase, which is suitable for use in continuous multiple stage processes. The mannose- or mannose-derivative-specific GDP-mannose-prophosphorylase, which can be isolated from microorganisms, has a specific activeity of≧2U/mg, is prepared.

The invention concerns a GDP-mannose-pyrophosphorylase. The aim of the invention is to produce a GDP-mannose-pyrophosphorylase which can be obtained for an acceptable outlay and does not cause problems, in particular because of its monofunctionality. in continuous multiple stage processes To that end, a mannose- or mannose derivative-specific GDP-mannose-pyrophosphorylase, which can be isolated from microorganisms and has a specific activity of≧2 U/mg, is prepared.

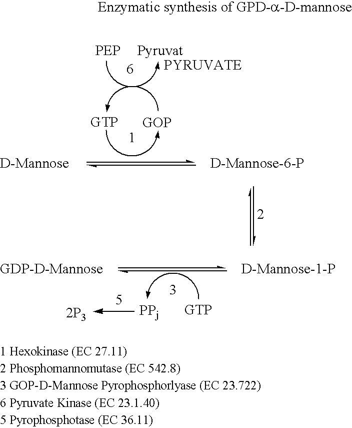

The object of the invention is a new GDP-mannose-pyrophosphorylase (GDPMan-PP) that is monofunctional with respect to the hexose residue, of microbial origin, which has a specific activity≧2 U/mg; and it comprises a method for the preparation of said enzyme as well as its use in the preparation of GDP-mannose.

GDP-mannose is one of the activated sugars that at this time have been extensively examined, and which can be reacted with glycosyl transferases to form oligosaccharides. Moreover, it forms the starting material for the preparation of GDP-fucose.

GDP-mannose pyrophosphorylase has been known for a long time. It has been isolated from various sources: in 1964 by Preiss et al. (J. Biol. Chem., Vol. 239, pp. 3119-26, 1964) via the isolation of the enzyme from Arthrobacter sp. D. Shinabarger et al. (J. Biol. Chem., Vol. 266, pp. 2080-88, 1991) describe the isolation of a multifunctional GDP-Man-PP from Pseudomonas aeruginosa with phosphomannose isomerase and pyrophosphorylase activity.

A GDP-Man-PP isolated from mammalian glands catalyzes both the synthesis of GDP-mannose and GDP-glucose. A CDP-Man-PP with 70,000-fold purification was prepared from porcine thyroid glands, which presented no GDP-glucose synthesis activity.

T. Szumilo et al. (J. Biol Chem., Vol. 268, pp. 17943-50, 1993) reports on the isolation and 5000-fold purification of GDP-Man-PP from porcine liver, where 4 mg of enzyme with a specific activity of 9.25 U/mg was isolated from 1 kg of liver in a multistep purification. This enzyme catalyzes both the formation of CDP-mannose and of GDP-glucose.

More recently, GDP-Man-PP-ouelle [sic; possibly quelle=source] has been obtained primarily from yeast (S. cerevisiae); as a rule, it is not subjected to a specific purification (P. Wang et al. in J. Org. Chem., Vol. 58, pp. 3985-90, 1993). In WO93/0820 Al, a report is made of the purification of GDP-Man-PP from yeast, where an enzyme solution with an activity of 0.1 U/mL was obtained from a yeast cell extract by fractionated (NH₄)₂SO4 precipitation and dialysis.

In summary it can now be observed that the commercially unavailable GDP-Man-PP is either isolated with very great expense or it is used in nonpurified or only partially purified form; also, and the forms of the enzyme differed, in part presenting multifunctionalities.

The goal of the invention therefore is a GDP-Man-PP that can be obtained for an acceptable expense and does not lead to problems, particularly because of its monofunctionality, in continuous multiple stage processes over longer periods of time.

The GDP-Man-PP developed for this purpose corresponds to claim 1. Other characteristics of the invention can be obtained from the secondary claim.

GDP-Man-PP is obtained in particular from a recombinant strain of microorganisms such as yeasts, B. subtilis, and E. coli strains as well as, possibly, from cell lines of animal origin, which are suitable for modification by genetic engineering to make producing strains and into which plasmids of the known type have been inserted, with these having been manipulated by genetic engineering to contain the gene coding for the desired formation of GDP-Man-PP, where the raw extract of the microorganisms contains the enzyme at a considerable concentration, so that the expense required for the preparation and purification for commercial production is entirely acceptable.

The table below shows the enzyme contents of the raw extract for different enzyme sources, the specific activity (to the extent known) obtained after purification, and the functionality of the enzyme produced.

Comparison of the enzyme source for GDP-mannose pyrophosphorylase

| | ²Spezifische | ³Spezifische | |
|---|---|---|---|
| | Aktivit\overline{a}ten | Aktivit\overline{a}t nach | ⁴Bemerkung zur |
| | Rohextrakt | Reinigung | Spezifit\overline{a}t bzw. |
| ¹Enzymquelle | (U/mg Protein) | (U/mg Protein) | Funktionalit\overline{a}t |
| ⁵Hefe (nach | 0.0167 | 1.32 | ⁶Mannose-1- |
| Munch-Petersen | | | Phosphat |
| 1962) | | | |
| ⁷Schweineleber | 0.0019 | 9.25 | ⁸(Glucose-1- |
| (nach Szumilo et | | | Phosphat und |
| al. 1993) | | | Mannose-1- |
| | | | Phosphat |
| ⁹Pseudomonas | 0.397 | 6.12 | ¹⁰Bifunkt\overline{i}onelles |
| aeruginosa | | | Enzym aus GDP- |
| (Shinabarger et | | | Man-PP und |
| al 1991) | | | Phosphomannose |
| | | | isomerase |
| ¹¹Escherichia coli | 0.0034 | | |
| (Wildstamm) | | | |
| eigene Messung | | | |
| mit NUSSA | | | |
| ¹²Rekombinanter E. | 0.370 | 2.3 | ¹³Mannose-1- |
| coli Stamm | | | Phosphat |
| erfindungsgem\overline{a}β | | | |

Key:
¹Enzyme source
²Specific activities Raw extract (U/mg protein)
³Specific activity after purification (U/mg protein)
⁴Remarks on the specificity or functionality
⁵Yeast (according to Munch-Petersen, 1962)
⁶Mannose-1-phosphate
⁷Porcine liver (according to Szumilo et al., 1993)
⁸(Glucose-1-phosphate and mannose-1-phosphate
⁹Pseudomonas aeruginosa (Shinabarger et al., 1991)
¹⁰Bifunctional enzyme from GDP-Man-PP and phosphomannose isomerase
¹¹Escherichia coli (wild strain) individual measurement with NUSSA
¹²Recombinant E. coli strain according to the invention
¹³Mannose-1-phosphate
Literature:
A. Munch-Petersen (1962), Meth. Enzymology, Vol. V, pp. 171-174, Szumilo et al. (1993), J. Biol. Chem. Vol. 268, pp. 17943-17950 Shinabarger et al. (1991), J. Biol. Chem., Vol. 266, pp. 2080-2088.

One can clearly see the superiority of the strategy, according to the invention, of preparing a productive enzyme cource of a monofunctional (“mannose-specific”) enzyme.

This enzyme can be used for the preparation of GDP-mannose in larger amounts, and it is advantageous here to start with the cheaper mannose-6-phosphate, which is first converted into mannose-1-phosphate using phosphomannomutase.

Both GDP-Man-PP and phosphomannomutase are obtained, particullarly starting from producing strains that contain the corresponding genes (rfbM or rfbK) after it has been inserted into a plasmid and the plasmid has been inserted in the corresponding producing strain, using the following strategy:

1. Amplification of the gene with PCR (vent [unconfirmed translation] polymerase)

After the chemical synthesis of primer based on known gene sequences, the genes are amplified by PCR with the vent polymerase.

2. Cloning of the gene in the plasmid pUC18 (blunt end with SmaI enzyme)

Cultivating in E. coli DH5α

After the separation of the amplified gene in an agarose gel and isolation of the gene from this gel, the genes are each ligated with a vector pUC18 (coupled) that has first been hydrolyzed with SmaI (restriction enzyme) for the blunt-end linearization. The ligated vectors are transformed in a strain of E. coli DH5α prepared for DNA uptake, then the cells are grown on a solid growth medium.

The positive transformed colonies (white colonies on the agar plate) are isolated and again grown as above.

3. Cloning of the gene in the expression vector pT7-6 using the EcoRI and BarnHI restriction sites

Cultivating in E. coli BL21(DE3)

From the positive transformants, the plasmid (plasmid pUC18+inserted gene rfbM or rfbK) is isolated, then hydrolyzed with the enzymes EcoRI and BarnHI. The expression vector pT7-6 is also hydrolyzed with the enzymes EcoRI and BamHI.

After the ligation of pT7-6 with the isolated gene rfbM or rfbK, a strain of E. coli BL21 (DE3), which has been prepared for DNA, is transformed with these genes. The transformants are grown on a solid nutrient medium.

After the isolation of individual colonies and renewed growing on a solid nutrient medium, each plasmid with the corresponding gene is isolated and hydrolyzed as a control.

The positive transformants are grown again on a solid nutrient medium and are subsequently stored.

Below, the invention is explained in further detail and with reference to specific embodiments.

The biosynthesis of activated sugar, particularly GDP-alpha-D-mannose, is, in vivo, often carried out starting with a monosaccharide (for example, mannose) that is phosphorylated at C6. The sugar-6-phosphate (for example, mannose-6-phosphate) is converted into a sugar-1-phosphate using a phosphomutase (EC 5.4), particularly in this instance a phosphomannomutase (EC 5.4.2.8).

Mannose-6-phosphate Mannose-1-phosphate (I)

Pyrophosphorylases belonging to the group of nucleotidyl transferases (EC 2.7.7), particularly in this instance the GDP-alpha-D-mannose pyrophosphorylase (EC 2.7.7.13), catalyze the transfer of a nucleotidyl group from a nucleoside triphosphate to form a sugar-1-phosphate with the release of inorganic pyrophosphate (see Feingold and Barber, 1990, in Methods in Plant Biochem, Vol. 2, pp. 39-78), particularly the following reaction

Mannose-1-phosphate+GTP GDP-Mannose+PP₁ (II)

Pyrophosphorylases make sugar nucleotides available as a substrate for glycosyl transferases (EC 2.4), which transfer the sugar portion to an acceptor (see, for example, Ginsberg, V. (1964) in Adv. Enzymol., Vol. 26, pp. 35-88, or for the synthesis of other secondary activated sugars, in this instance particularly GDP-β-L-fucose (Yamamoto, K., 1982, Agric. Biol. Chem., Vol. 48, pp. 823-824 and 1993, Arch. Biochem. Biophys., Vol. 300, pp. 694-698).

Since the chemical synthesis is often difficult and is associated with low yields, enzymatic synthesis is increasingly being used.

The phosphomannomutase (EC 5-4.2.8) and the GDP-mannose-pyrophosphorylase (EC 2.7.7.13) have, so far, been detected in different sources.

GDP-mannose pyrophosphorylase was partially isolated for the first time in 1956 by Munch-Petersen from baker's yeast, with considerable variations in the quantity of available enzyme depending on the yeast load (Munch-Petersen, 1956, Acta Chem. Scand., Vol. 10, p. 928). The enzyme was isolated in 1962 by Preiss and Wood (J. Biol. Chem., Vol. 239, No. 10, pp. 3119-3126) from Arthorobacter sp. However, the authors were unable to rule out that the numerous reacted activated sugars were the result of secondary reactions of other pyrophosphorylases. In Pseudomonas aeruginosa and Rhodospirillum rubrum a bifunctional enzyme has been found, GDP-mannose-pyrophosphorylase, coupled with phosphomannose-isomerase activity (Shinabarger et al., 1991, J. Biol. Chem., Vol. 266, No. 4, pp. 2080-2088 and Ideguchi et al., 1993, Biochimica et Biophys. Acta, Vol. 1172, pp. 329-331). From the eukaryotic species as well, GDP-mannose-pyrophosphorylase has been isolated (Szumilo et al., 1993, J. Biol. Chem., Vol. 268, No. 24, pp. 17943-17950). The activities that were determined—conversion to GDP-glucose (100%), IDP-glucose (72%), and GDP-mannose (61%)--suggest that this pyrophosphorylase is instead a GDP-glucose-pyrophosphorylase (EC 2.7.7.34).

So far, the phosphomannomutase (EC 5.4.2.8) has only been considered in connection to alginate biosynthesis (Sa-Correia et al., 1987, J. Bacteriol., Vol. 169, pp. 3224-3231 and Goldberg et al., 1993, J. Bacteriol., Vol. 175, No. 3, pp. 1605-1611).

The enzymatic synthesis of GDP-mannose has until now been described by Simon et al., 1990, in J. Org. Chem., Vol. 55, pp. 1834-1841, Wong et al., 1993, in WO 93/0820, Wang et al., 1993, in J. Org. Chem., Vol. 58, pp. 3985-3990, and Palanka and Turner, 1993, in J. Chem. Soc. Perkion Trans., Vol. 23, No. 1, pp. 3017-3022. These work groups all use a protein preparation obtained, according to a method described by Munich-Petersen in 1956, from yeast cells, and they synthesize GDP-mannose starting with mannose-1-phosphate prepared by a chemical route.

By cloning in a (production) expression vector (plasmid) (pT7-6 from the Novagen company) and (insertion) transformation in a (production strain) expression strain Escherchia coil BL21(DE3)pLysS (from the Novagen company), a phosphomannomutase and GDP-mannose pyrophosphorylase were then developed; this, according to the invention, can be obtained in larger quantities than from the sources known so far (see Table, page 3). Both enzymes originate from Salmonella enterica, group B (formerly Salmonella typhimurium LT2). The genes (rfbM codes for the GDP-mannose pyrophosphorylase; rfb K codes for the phosphomannomutase) are located in the rfb gene cluster whose structure and sequence has been elucidated by Jiang et al., 1991, in Mol. Microbiol., Vol. 5, No. 3, pp. 695-713.

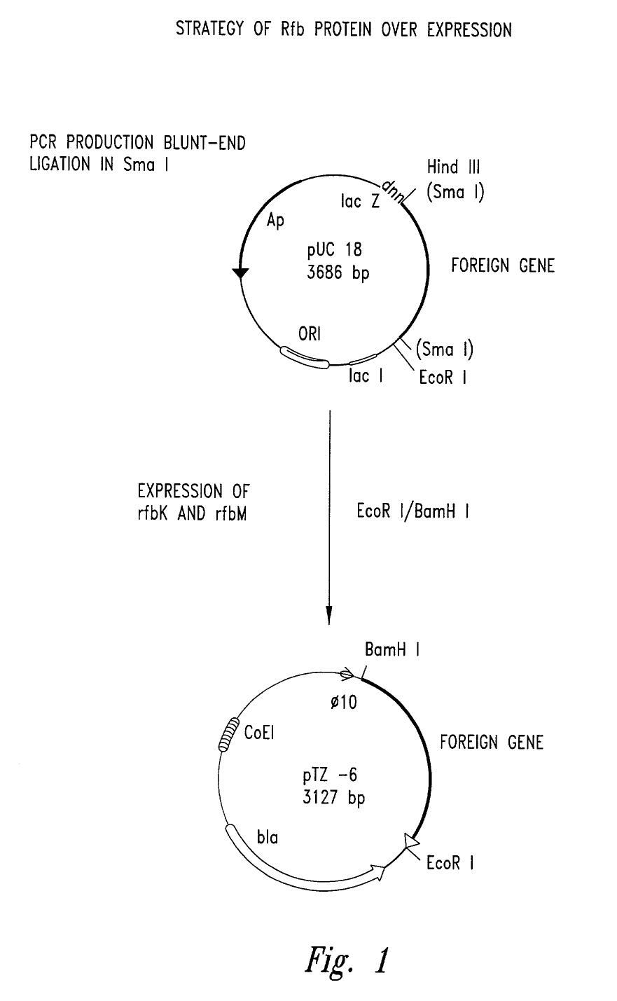

Using the polymerase chain reaction (PCR) the genes rfb M and rfb K are multiplied (amplified) and each is cloned in a vector pUC18 (Novagen company). Starting with this vector, the genes rfb M and rfb K are each cloned in an expression vector pT7-6 from the Novagen company, and each is inserted (transformed) in an expression strain of Escherichia coli BL21(DE3)pLysS. The plasmid pT7-6 with the inserted gene rfb M is now called pERJ-1. The plasmid pT7-6 with the inserted gene rfb K was named pERJ-2 (see FIGS. 1-3).

The production (expression) of the proteins (GDP-mannose pyrophosphorylase and phosphomannomutase), coded by the genes rfb M and rfb K, was induced with 0.4mM isopropyl thiogalactoside (IPTG) and amplified with 0.03mM rifampicin (see FIG. 4).

By mechanical breakup of the cells (Escherichia coli) in 50mM Tris-HCl buffer, pH 8, and 150mM KCl and centrifugation (2 min at 10,000 rpm), a protein-containing raw extract was obtained. This raw extract was loaded in an anionic exchanger (Q-Sepharose FF), and the GDP-mannose pyrophosphorylase was obtained by an incremental elution with 150 mM KCl and 400 mM KCl. The eluate was reacted with 1M ammonium sulfate and 20% glycerin (v/v), and applied to a phenyl Sepharose FF. After adsorption, the enzyme is eluted with a gradient between 1M ammonium sulfate and OM ammonium sulfate in 50M Tris-HC1, pH 8, 20 [sic] glycerin between 0.4M and 0.1M ammonium sulfate. After ultrafiltration and a buffer change with 50 mM Tris-HC1, pH 8, with 150 mM KCl, the GDP-mannose pyrophosphorylase was chromatographed on a gel filtration column.

After ultrafiltration, the GDP-mannose pyrophosphorylase is reacted with 3M ammonium sulfate and stored at 4° C.

The phosphomannomutase should be partially purified on a Q Sepharose FF column.

Both in the case of phosphomannomutase and in the case of the GDP-mannose pyrophosphorylase, the enzymes are monofunctional and specifically catalyze the reactions described for them (see I and II above).

Both enzymes should be used for the enzymatic synthesis of GDP-mannose, starting with mannose, according to the following reaction schemes:

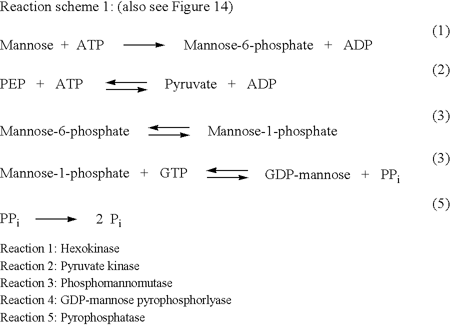

Reactions 1 and 2 have already been used for the production of mannose-6-phosphate by Palanca and Turner, 1993, J. Chem. Soc. Perkin Trans., Vol. 1, pp. 3017-3022.

The GDP-mannose formed in this manner can be further reacted in situ with mannosyl transferase to form oligosaccharides.

The GDP-mannose pyrophosphorylase activity was demonstrated with a newly developed continuous spectrophotometric test for the determination of pyrophosphate (PP₁) producing nucleotidyl transferases (EC 2.7). In the manner described below, it is possible to determine, by the use of the substrates (sugar-1-phosphates or sugar in the case of neuraminic acid) and nucleoside triphosphates any pyrophosphorylase activity which presents an activity in the test mixture of≧0.2 mU/mL.

The enzyme test (Nucleotidyl transferase substrate screening assay ‘NUSSA’) is based on the fact that in the nucleotidyl transferase reaction (EC 2.7-7), pyrophosphate is produced with a pyrophosphate-dependent phosphofructokinase (PPᵢPFK from plants or bacteria EC 2.7.1.90) with fructose-6-phosphate and in the presence of fructose-2,6-diphosphate to make fructose-1,6-diphosphates. This product is cleaved with an aldolase to form dihydroxyacetone phosphate (DHAP) and glycerin-3-phosphate (GAP). From glycerinaldehyde-3-phosphate, dihydroxyacetone phosphate is produced with the triose phosphate isomerase. Finally, dihydroxyacetone phosphate is reduced with glycerin-3-phosphate dehydrogenase to glycerin-3-phosphate (G-3-P) with NADH. 2 mol of NADH are used per mol of pyrophosphate; this consumption can be monitored by photometry.

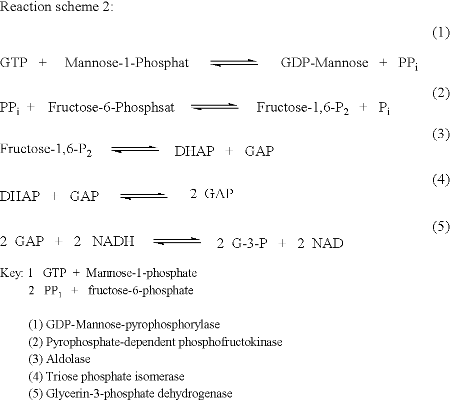

The following examples present the protocol according to the invention in detail. Reference is made here to the drawings in the appendix, where the figures represent:

FIG. 1: the cloning strategy

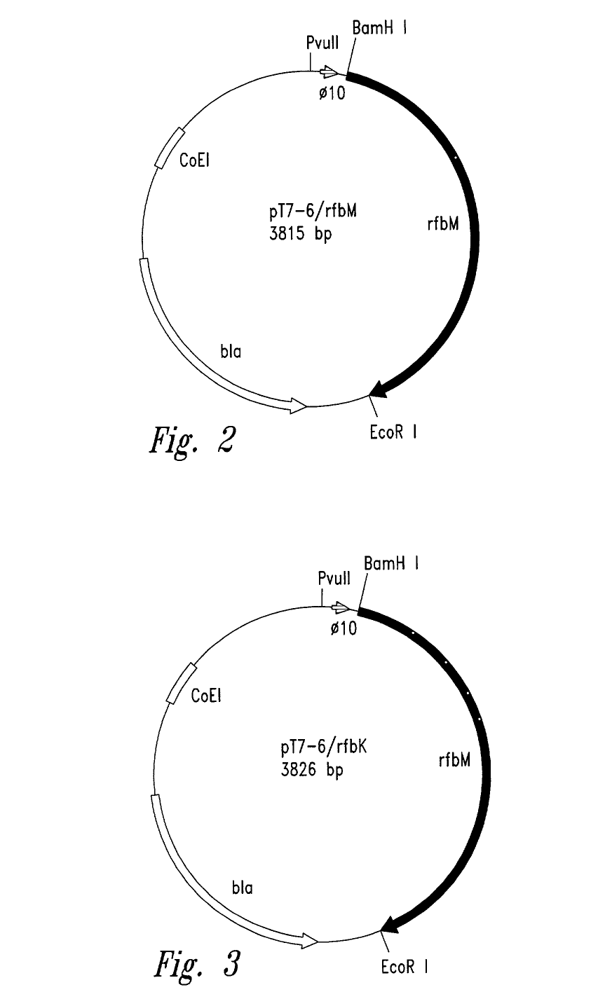

FIG. 2: the expression vector pERJ-1

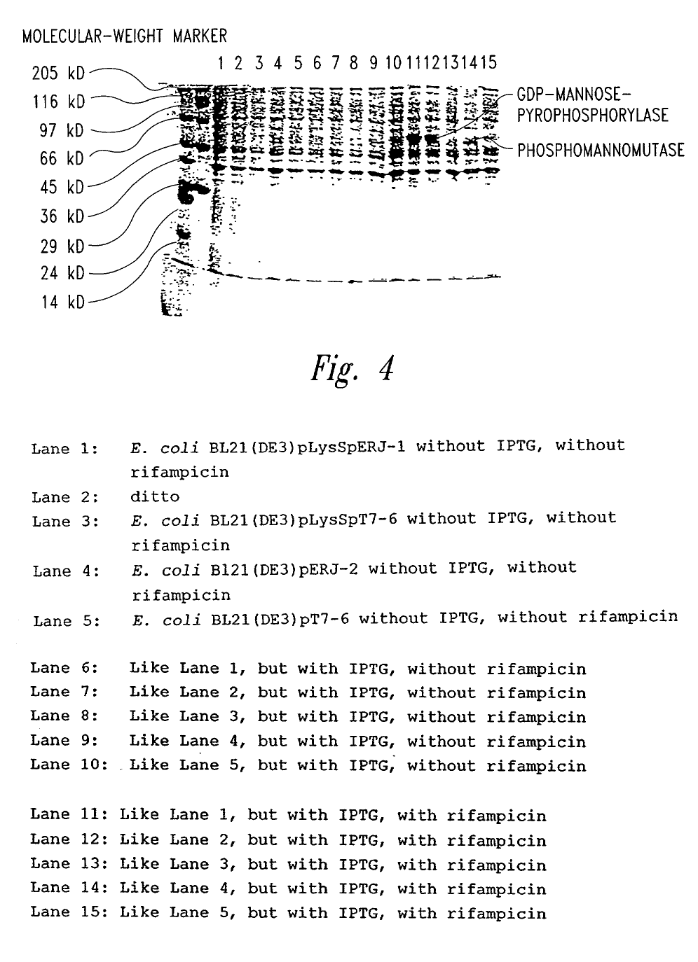

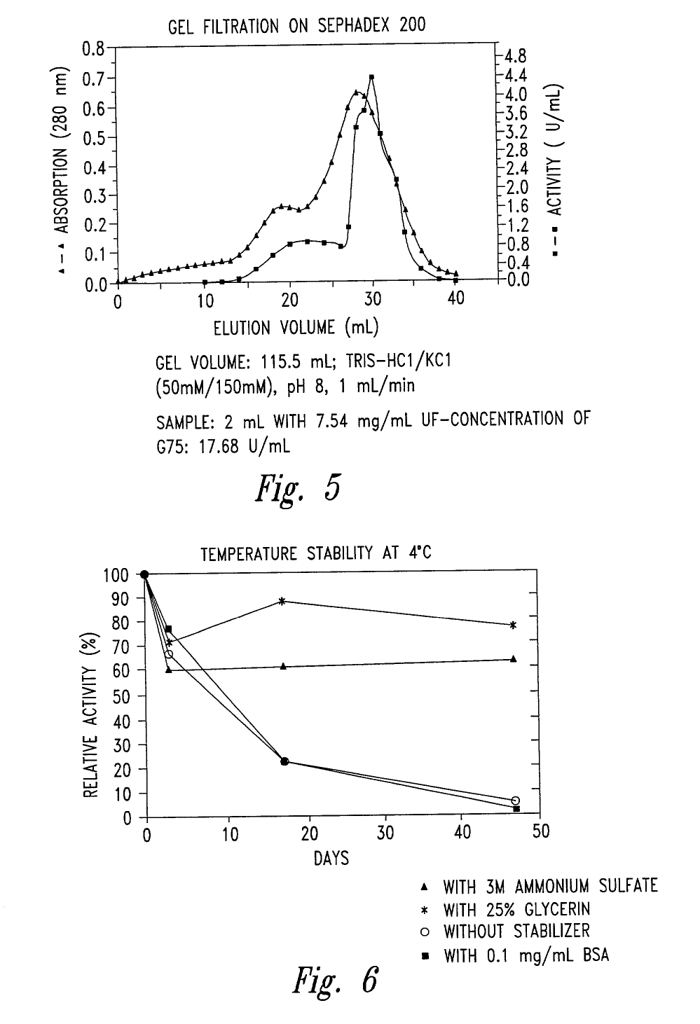

FIG. 3: the expression vector pERJ-2

FIG. 4: the SDS-gel electrophoresis of the expressed gene products of pERJ-1 and pERJ-2

FIG. 5: chromatogram of the gel filtration for the determination of the molecular weight

FIG. 6: stability of a GDP-man-pyrophosphorylase at 4° C.

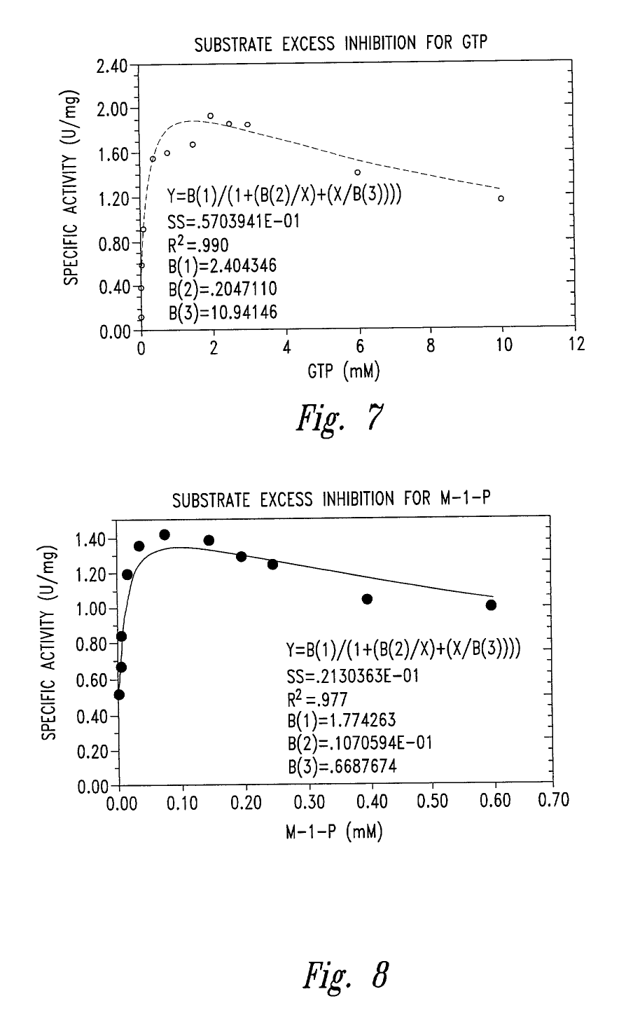

FIG. 7: the substrate excess inhibition of GTP

FIG. 8: the substrate excess inhibition of M-1-P

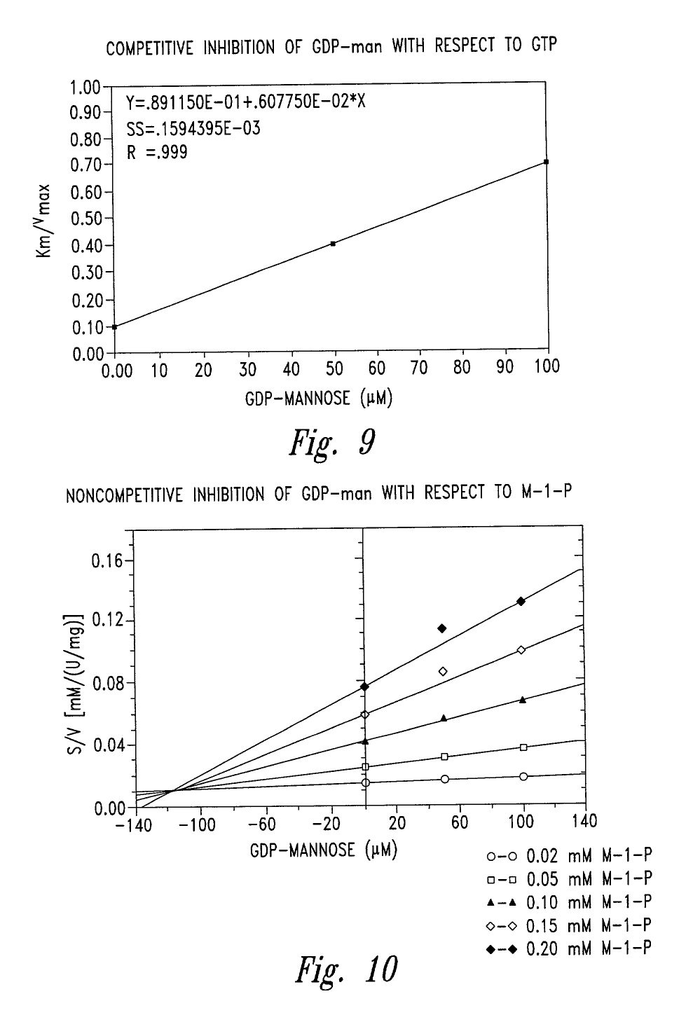

FIG. 9: the competitive inhibition of GDP-Man with respect to GTP

FIG. 10: the noncompetitive inhibition of GDP-Man with respect to M-1-P

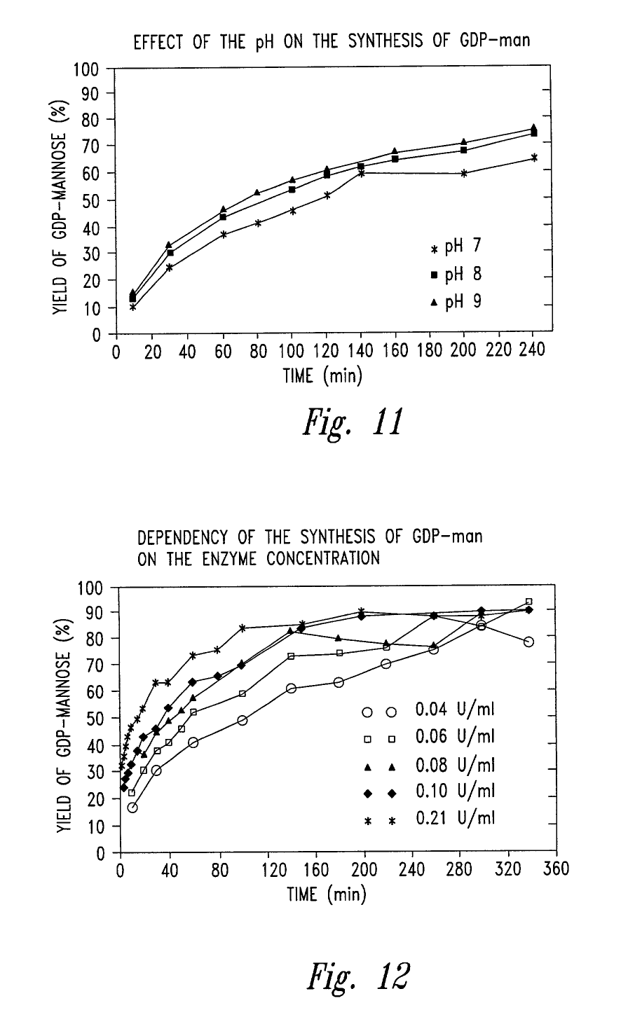

FIG. 11: the influence of the pH on the synthesis of GDP-mannose

FIG. 12: the dependency of the synthesis of GDP-mannose on the enzyme concentration

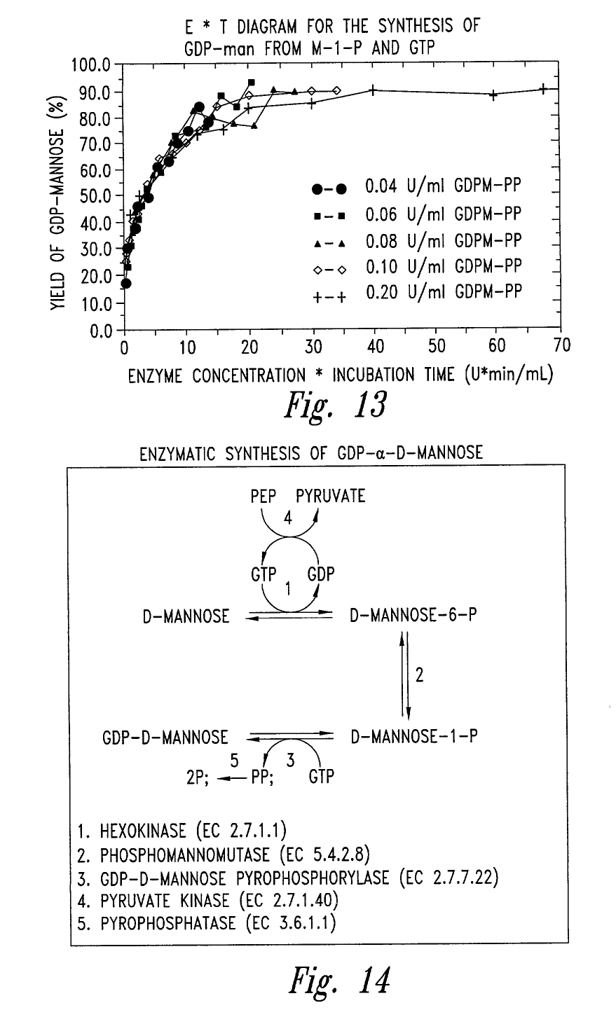

FIG. 13: the E * t diagram for the synthesis of GDP-man starting with mannose-1-phosphate and GTP

FIG. 14: reaction scheme of the biosynthesis of GDP-mannose from mannose

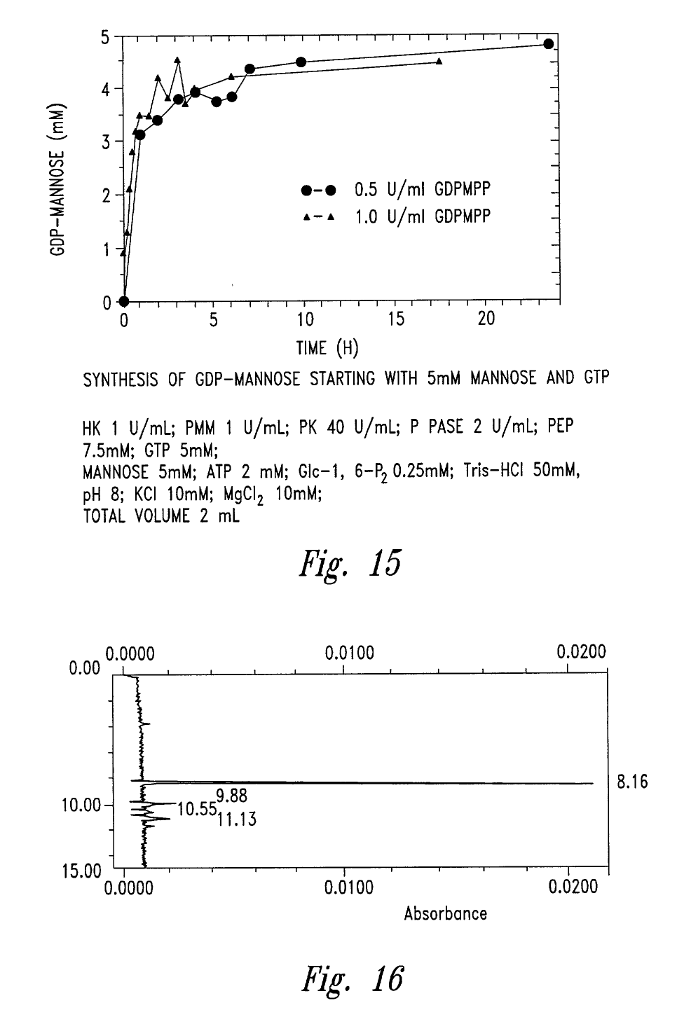

FIG. 15: synthesis of GDP-mannose starting with 5 mM mannose

FIG. 16: capillary electrophoresis chromatogram of the GDP-mannose prepared

### EXAMPLE I

Cloning of the genes rfb M and rfb K from the rfb gene cluster of Salmonella enterica, group B.

Using a DNA data bank, the genes rfb M and rfb K were identified in the rfb gene cluster, and the reading frame was determined.

rfb M: codes for the GDP-alpha-D-mannose pyrophosphorylase (EC 2.7.7.13)

Length in Bp: 17386-18831: 1445 base pairs

Start codon: ATG 17386

Stop codon: TAA TAA TAG 18831

Ribosome binding site: AAA AGA GAT AA

rbf K codes for phosphomannomutase (EC 5.4.2.8)

Length in Bp.: 18812-20245: 1433 base pairs

Start codon: ATG 18812

Stop codon: TAA 20245

Ribosome binding site: GAA GGA GTG GA

For the in vitro amplification, the following oligonucleotide primers for both genes were determined.

| | | |
|---|---|---|
| rfb M: | | |
| Primer 1: | (rfb M1) | 5′-CTT GGG TTA CAA ATT AGG CA-3′ |
| Primer 2: | (rfb M2) | 3′-ATC TTT TAC AAG ACC GCG AG-5′ |
| rfb K: | | |
| Primer 1: | (rfb K1) | 5′-CCC CCT GAA GTT AAT TGA GA-3′ |
| Primer 2: | (rfb K2) | 3′-CCA TTT AAT CCT CAC CCT CT-5′ |

The length of the gene is thus increased for rfb M to 1633 Bp. and for rfb K to 1606 Bp.

The PCR is carried out as follows:

TABLE I
PCR preparation for the cloning of rfb M and rfb K

| | rfb M | rfb K |
|---|---|---|
| ¹Vent-Polymerase | 1 μl (2U) | 1 μl |
| ²Vent-Polymerase-Puffer (10x) | 10 μl | |
| H₂O | 54.4 μl | 52.1 μl |
| ³dATP, dCTP, dGTP, | 16 μl | |
| dTTP je 1.25 mM | | |
| Primer 1 | | |
| rfb M1 | 6.2 μl | |
| | (23 pmol/μl) | |
| rfb K1 | | 6.1 μl |
| | | (23.6 pmol/μl) |
| Primer 2 | | |
| rfb M2 | 7.4 μl | |
| | (19.4 pmol/μl) | |
| rfb K2 | | 9.9 μl |
| | | (14.5 pmol/μl) |
| ⁴genomische DNA aus Salmonella | 5 μl | 5 μl |
| (μ 2 μg/100 μl) | | |
| MgCl₂ (25 mM) | 10 μl | 10 μl |

Key:
¹Vent polymer
²Vent polymer buffer (10x)
³dATP, dCTP, dGTP, dTTP, each at 1.25 mM
⁴Genomic DNA from Salmonella

The preparations are covered with 70 μL of mineral oil each, to prevent evaporation.

Vent polymerase buffer (BioLabs, New England) (10x) 200 mM Tris-HCl, pH 8.8, 100 mM KCl, 100 mM (NH₄)₂SO₄, 20 mM MgSO₄, 1% Triton 100X (w/v)

The following conditions were selected to run the PCR:

| | | |
|---|---|---|
| 5 min | 98° C. | |
| 2 min | 95° C. | repeat 6 times |
| 30 sec | 49° C. | |
| 90 sec | 72° C. | |
| 1 min | 95° C. | |
| 45 sec | 49° C. | repeat 25 times |
| 90 sec | 72° C. | |
| 2 min | 72° C. | |
| Cool | | |

After the PCR, the amplified genes, each 1.6 kB, were isolated according to Lau and Sheu, 1992, Meth. Mol. Cell. Biol., Vol. 3, pp. 190-192, from an agarose gel, also, each was ligated in an auxiliary vector pUCi8, which has first been “blunt ended” linearized using the restriction enzyme SmaI. The ratio of the vector to the DNA fragment was approximately 1:4. The ligation was carried out overnight at 14° C.

TABLE II
Preparations for the ligation of the PCR products in pUC18

| | | μL | |
|---|---|---|---|
| T4 ligase | | 1 | |
| Ligase buffer (10x) | | 6 | |
| Vector pUC18/SmaI | | 1 | 20-60 μg/preparation |
| H₂O (sterile) | rfb M | 12 | |
| | rfb K | 18 | |
| DNA fragment | rfb M | 10 | 80-240 μg/preparation |
| | rfb K | 4 | |

Ligase buffer (10x): 0.5 M Tris-HC1, pH 7.6, 100 mL MgCl₂, 100 mM DTT, 500 μg/mL BSA

This vector with the inserted gene was transformed in competent cells of Escherichia coli DH5alpha (according to Hanahan, 1983, J. Mol. Biol., Vol. 166, pp. 557-580). For this purpose, 5 μL of the ligation preparations and 15 μL of sterile H₂O were each reacted with 200 μL of competent cells that had been thawed on ice, then incubated for 30 min on ice. The preparations were then heated for 40 sec at 42° C., and again placed on ice for 2 min. 800 μL of SOC medium were then added to the preparations; the preparations were then incubated for 1 h at 37° C., then spread on LB_{Amp-100} agar plates, which were coated with X-Gal.

SOC medium: pH 7, 20 g tryptone, 5 g yeast extract, 0.5 g NaCl, 2.5mM KCl, 10 mM MgCl₂, 20 mM (filtration sterilized) glucose, H₂O and 1000 mL

Lb_{amp-100: 10} g tryptone, 5 g yeast extract, 5 g NaCl (and 15 g Bacto agar)

Ampicillin 100 mg/L

X-Gal: 5-Bromo-4-chloroindolyl-β-D-galactose (40 mg/mL) 70 μL per agar plate

The plasmid pUC18/rfb M or rfb K was isolated from the positive colorless colonies (according to Birnboim and Doly, 1979).

pUC18/rfb M was linearilized with the restriction enzymes EcoRI and BamHI; the gene rfb M was cut out of the vector. From an agarose gel, the genes rfb M and rfb K were isolated, then ligated in the expression vector (pT7-6).

TABLE III
Preparations for the ligation of the genes rfb M and rfb K in pT7-6

| | | μL | |
|---|---|---|---|
| T4-ligase | | 1 | (0.1 Weiss unit) |
| T4-ligase buffer (10x) | | 6 | (see above) |
| Vector pT7-6/Eco RI-BamHI | | 2 | |
| H₂O sterile | rfb M | 13 | |
| | rfb K | 17 | |
| DNA | rfb M | 8 | |
| | rfb K | 3 | |

These ligation preparations (pT7-6/rfb M and pT7-6/rfb K) are transformed in competent cells (according to Cohen, Shng [sic], and Hsu, 1972, Proc. Nat. Acad. Sci. (USA), Vol. 69, No. 8, pp. 2110-2114) with, in the case of rfb M, Escherichia coli BL21(DE3)pLysS from the Novagen company, and, in the case of rfb K, in Escherichia coli BL21(DE3).

The strain that contains the gene rfb M inserted in pT7-6 will be called E. coli BL21(DE3)pLysSpERJ-1 below. The strain that contains the gene rfbk inserted in pT7-6 will be called E. coli BL21(DE3)PERJ-2 below.

The expression of the genes rfb M and rfb K is carried out as follows:

From preliminary cultures of Escherichia coli BL21(DE3) pLysSpERJ-1 (5 mL LB_{Amp-Chloramp-50} (ampicillin and chloramphenicol, each 50 mg/L) overnight at 120 rpm and 37° C.), main cultures (10 mL) were inoculated at 2% and they were cultured in Erlenmeyer flasks with baffles at 37° C. and 120 rpm with a shaker until an optical density of 0.5, at 546 nm, was reached; 1 mL of the culture was removed, then centrifuged, with the supernatant being removed and the pellet reacted with 50 gL of sample buffer (SDS and containing β-mercaptoethanol). This sample was heated for 3 min at 95° C. and was placed on an SDS polyacrylamide gel (method, see below). The rest of the culture was reacted with 0.4 mM IPTG and incubated for 20 min, then 1 mL was again removed and treated as above. The rest of the culture was reacted with 0.03 mM rifampicin and incubated for 60 min; 1 mL was removed and treated as described above; 10 μL of each of these samples was applied onto an SDS-polyacrylamide gel.

Escherichia coli BL21(DE3)pERJ-2 was cultured as described above.

An SDS-polyacrylamide gel electrophoresis was carried out according to Laemmli, 1970, Nature, Vol. 227, pp. 680-685. A photograph of the Coomassie brilliant blue stained gel is shown in FIG. 4.

Isolation of the GDP-mannose pyrophosphorylase from Escherichia coli BL21

Culturing of E. coli BL21 and breakup of the cells:

at 37° C. in a shaker at 120 rpm

Starting with a preliminary culture (overnight incubation, 200 mL in a 1000-mL Erlenmeyer flask with baffles), 2 L of LB_{Amp-Chlorap-50} were placed in each of five 5-L flasks with baffles and inoculated at 1%; the cultures were grown until the optical density was 0.8 (3.5 h). After a 20-min incubation with 0.4 mM IPTG and a 60-min incubation with 0.3 mM rifampicin, the cultures were removed by centrifugation (Sorvall GS3, 8000 rpm, 10 min, 20° C.) and washed twice with 50 mM Tris-HCl, pH 8. The wet weight was then determined (approximately 25 g) and a 20% (w/v) cell suspension was prepared. The cells were then broken up in a disintegrator S by wet grinding. For this purpose, 40 g of cell suspension were mixed with 80 g of glass beads (0.3 mm diameter) and homogenized for 12 min at 4000 rpm. The cell debris and the glass beads were separated by a 15-min centrifugation (Sorvall GSA, 10,000 rpm, 20° C.), washed in 50 mM Tris-HCl, pH 8, and centrifuged again. The supernatants were cleaned; they formed the raw extract for the anion exchange chromatography on Q-Sepharose FF. Q-Sepharose FF:

400 mL of Q-Sepharose FF were loaded with 226 mL of raw extract (with 11.1 mg/mL of protein). The stepwise elution starts with approximately 800 mL 50mM Tris-HCl, pH 8, and approximately 1400 mL of 50mM Tris-HCl, pH 8, with l5OmM KC1. The enzyme is eluted with approximately 900 mL of 50mM Tris-HCl, pH 8, with 400 mM KCl. This fraction is reacted with 1M ammonium sulfate and 20% glycerin, and is loaded onto phenyl Sepharose FF (66 mL). The enzyme is eluted using a gradient that decreases linearly to OM ammonium sulfate, with 50mM Tris-HCl, pH 8, and 20% glycerin (total volume 1000 mL). The most active fractions, between 0.4M and 0.1M ammonium sulfate, were purified; the buffer was changed after ultrafiltration (50 mM Tris-HCl, pH 8, 150 mM KCl), then chromatographed in a gel filtration column (Superdex G-75) (see Table IV below).

The recombinant GDP-mannose pyrophosphorylase was concentrated by 6.3 times from E. coli. With a yield of 13.5%, a specific activity of 2.34 U/mg could be obtained. Starting with 0.37 U/mg, it was possible to achieve a purification factor of 2 using Q-Sepharose, with a yield of 85%. The subsequent hydrophobic interaction chromatography on phenyl Sepharose led, as a result of the combination of only the most active fractions, to a relatively high loss of 40%, with an increase in the specific activity to 2.27 U/mg.

The molecular weight of the GDP-mannose pyrophosphorylase was, under denaturing conditions (SDS-polyacrylamide gel electrophoresis), 54 kD. For the determination of the molecular weight in the native state, with 2 mL (7.54 mg/mL) of enzyme sample from the purification, see above, a gel filtration was performed on Sephadex G-200 (115.5 mL). The determination of the activity was carried out using the enzyme test, according to the invention, for phosphorylases, as described below. Two activity maxima were determined, which correspond to the molecular weights of 208,700 dalton and 107,800 dalton. In the native state the enzyme was thus in the form of a dimer or a tetramer.

TABLE IV
Purification of the GDP-mannose pyrophosphorylase;
results of the individual purification steps

| | Gesamt- | Gesamt- | Spezifisebe | Reini- | |
|---|---|---|---|---|---|
| | protein | aktivit\overline{a}t | Aktivit\overline{a}t | gungs- | Ausbeu\dot{t}e |
| Probe¹ | [mg]² | [U]³ | [U/mg]⁴ | faktor⁵ | [%]⁶ |
| Rohextrakt⁷ | 2504 | 918.1 | 0.37 | 1.0 | 100 |
| Q-Sepharose FF | 981 | 782.3 | 0.79 | 2.1 | 85.2 |
| HIC | 153 | 347.2 | 2.27 | 6.1 | 37.8 |
| (Phenylseph.)⁸ | 126 | 270.0 | 2.14 | 5.8 | 29.4 |
| UF (HIC) | 53 | 123.7 | 2.34 | 6.3 | 13.5 |
| C-75 UF | | | | | |
| (Gel filtration)⁹ | | | | | |

Key:
¹Sample
²Total protein (mg)
³Total activity (U)
⁴Specific activity (U/mg)
⁵Purification factor
⁶Yield (%)
⁷Raw extract
⁸(Phenyl Sepharose)
⁹(Gel filtration)

The following examinations were carried out on the method of action and the use of GDP-alpha-mannose pyrophosphorylase:

1) Examinations of stability

The enzyme was examined at 4° C. to determine its stability during storage. For this purpose, an enzyme preparation was reacted with 1.15 U/mg without a stabilizer, and with 0.1 mg/mL BSA, 3M ammonium sulfate, or 25% glycerin, then reacted for 47 days at 4° C. After 47 days, a residual activity of approximately 5% could be found in the preparations without a stabilizer and with BSA, whereas the preparations with glycerin and ammonium sulfate still presented activities of 75% and 65%, respectively. In the preparation with ammonium sulfate it was possible to determine an activity of 50% of the starting activity even after 4 months (FIG. 6).

Furthermore, the stability at 30° C. was examined by incubating a defined stock solution of enzyme with 79.3 mU/mg at 30° C. in 50 mM Tris-HCl, pH 8, 5 mM MgCl₂, and an activity determination was performed at different times (0 h, 2 h, 6 h, and 30 h).

TABLE IVa
Temperature stability of GDP-man-pyrophosphorylase at 30° C.

| ¹Stunden [h] | ²Aktivit\overline{a}t (mU/mgl | ³Relative Aktivit\overline{a}t [%] |
|---|---|---|
| 0 | 79.3 | 100 |
| 2 | 78.4 | 98.9 |
| 6 | 67.8 | 85.6 |
| 30 | 53.7 | 67.7 |

⁴Aktivit\overline{a}tsbestimmung per NUSSA mit 2 mM GTP und
0.08 in mM M-1-P
Key:
¹Hours (h)
²Activity (mU/mg · L)
³Relative activity (%)
⁴Activity determination by NUSSA with 2 mM GTP and 0.08 mM M-1-P

2) Determination of the kₘ and vₘₐₓ values for the substrates GTP and mannose-i-phosphate (M-1-P)

Conditions: 2.27 μg/μL of GDP-mannose pyrophosphorylase preparation after the gel filtration, with activity determination be NUSSA.

a) In the determination of the kₘ value and the vₘₐₓ value for GTP mannose-1-phosphate, a constant concentration of 0.08 mM was used. GTP was used at concentrations of 0.01-10 mM (FIG. 7).

b) In the determination of the kₘ value and the vₘₐₓ value for M-1-P GTP, a constant concentration of 2 mM was used. Mannose-1-phosphate was used at a varying concentration of 0.002-0.6 mM (FIG. 8).

TABLE V
Kinetic constants for the substrates GTP and
M-1-P of the GDP-mannose pyrophosphorylase

| | GTP | M-1-P |
|---|---|---|
| kₘ value | 0.2 mM | 0.01 mm |
| kₘₐₓ value | 2.4 U/mg | 1.8 U/mg |
| kᵢ value (excess substrate) | 10.9 mM | 0.7 mM |

3) Influence of GDP-mannose on the synthesis

Conditions: 2.27 μg/mL (in a) and 5.67 μg/mL (in b) GDP-mannose pyrophosphorylase preparation after gel filtration Activity determination by NUSSA

a) Mannose-1-phosphate was used at a constant concentration of 0.08 mM.

The concentration of GTP was varied between 0.08mM and 6 mm.

The GDP-mannose was used at 0μM, 50 μM, and 100 μM in the test.

The evaluation of the measured activities shows a competitive inhibition of GDP-mannose with respect to GTP (FIG.

9). The calculated Kᵢ value was 14.9 μM.

b) GTP was used at a constant concentration of 2 mM in the test.

Mannose-1-phosphate was used at varying concentrations of 0.003-0.3 mM.

The evaluation of the measured activities showed a noncompetitive inhibition of GTP-mannose with respect to M-1-P (FIG. 10). The calculated Kᵢ value was 118 μM.

4) Substrate spectrum of the GDP-mannose-pyrophosphorylase

Conditions: The GDP-mannose pyrophosphorylase was used at a concentration of 7.3 mU/mg in the NUSSA enzyme test.

a) Nucleoside triphosphates: ATP, CTP, GTP, UTP, dTTP, each lmM sugar-1-phosphate: mannose-1 -phosphate at 2.5 mM.

b) Nucleoside triphosphate: GTP

Sugar-1-phosphates: Glucose-1-P, N-acetylglucosamine-1-P-, glucosamine-1-P, galactose-1-P, galactosamine-1-P, N-acetylgalactosamine-1-P, glucuronic acid-1-P, galacturonic acid-1-P, xylose-1-P, mannose-1-P

Both in a) and in b), no reaction could be determined except with the natural substrates GTP and mannose-1-phosphate.

5) Use of the GDP-mannose pyrophosphorylase for the synthesis of GDP-mannose

The synthesis should be carried out starting with mannose and using reaction scheme 1.

First, the synthesis of GDP-mannose was examined, starting with mannose-1-phosphate and GTP. For this purpose, the GDP-mannose pyrophosphorylase was used in a form coupled to the pyrophosphatase (1 U/mL).

The synthesis was carried out at different pH values (7, 8, 9) in 50 mM Tris-HCl, 5 mM MgCl₂ with 2 mM GTP, and 2mM mannose-1-phosphate in a total volume of 2 mL at room temperature. The GDP-mannose pyrophosphorylase was used at 0.04 U/mL and the pyrophosphatase was used at 1 U/mL. After different times, 200 μL were removed from the preparation and heated for 5 min at 95° C., followed by centrifugation (Eppendorf centrifuge, 10,000 rpm, 2 min, room temperature) and analysis by capillary electrophoresis.

Using calibration curves and a comparison of the areas under the curves, the content of GTP and GDP-mannose could be determined. The reactions show a higher yield of GDP-mannose at alkaline pH values (FIG. 11). Since the other auxiliary enzymes (reaction scheme 1: hexokinase and pyruvate kinase) have optimum pH values of 7-9 (Boehringer-Mannheim, 1987, in Biochemica-Information) a pH of 8 was selected for additional syntheses.

Below, the dependency of the synthesis of GDP-mannose on the enzyme concentration of GDP-mannose pyrophosphorylase was examined, with the latter being used at 0.04 U/mL, 0.06 U/mL, 0.08 U/mL, 0.1 U/mL, and 0.2 U/mL. The reactions show an increased yield of GDP-mannose with the same incubation times and increased enzyme concentrations (FIG. 12). The multiplication of the enzyme concentration by the incubation time leads to a reaction constant (E * t). If the enzyme concentration or the incubation time is changed, constant yields can be obtained if the E * t product is maintained constant. For an E * t of 20 (U*min/mL), the reaction equilibrium is established under selected conditions, reaching a yield of GDP-mannose of approximately 90% (FIG. 13).

### EXAMPLE II

Nucleotidyl transferase substrate assay (NUSSA)

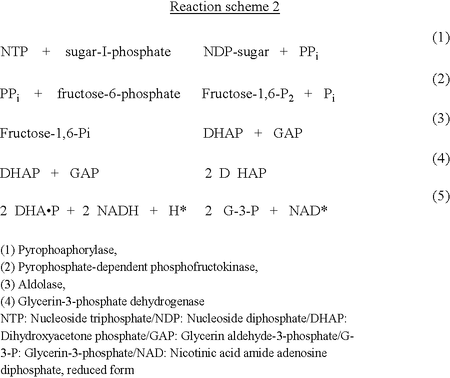

O'Brien, Bowien, and Wood described in 1975, J. Biol. Chem., Vol. 250, No. 22, pp. 8690-8695, a coupled photometric enzyme test for measuring a pyrophosphate-dependent phosphofructokinase (PP₁PFK), which was discovered for the first time by Reeves et al., 1974, J. Biol. Chem., vol. 249, pp. 7737-7741, in Entamoeba histolytica. In this measurement, the PPIPFK was coupled with the reaction of the aldolase (reaction 3), the triose phosphate isomerase (reaction 4), and the glycerin-3-phosphate dehydrogenase (reaction 5). The reaction was monitored by photometry at 340 nm.

The following enzyme test (NUSSA) coupled, according to the invention, the reaction of the nucleotidyl transferase with this test system; it thus makes it possible to measure any pyrophosphorylase or any pyrophosphate-releasing enzyme. The NUSSA test was optimized for measurement in microtiter plates with a total volume of 200 μL.

TABLE VI
Composition of the NUSSA enzyme test

| | | | Pyrophosphorylase- |
|---|---|---|---|
| | ¹Endkonzentration | ²PP₂PFK-Test | Test |
| Tris-HCl, pH 8 | 50 mM | | |
| MgCl₂ · 6 H₂O | 5 mM | 128 μl | 108 μ ^{±} × μl |
| NADH | 0.15 mM | 10 μl | 10 μl |
| ⁴Fructose-6-Phosphat | 2.5 mM | 10 μl | 10 μl |
| Fructose-2, 6-P₂ | 1 μM | 10 μl | 10 μl |
| PP₂ | 2.5 mM | 10 μl | — |
| ⁵Zucker-1-Phosphat | variabel | — | 10 μl |
| ⁶Nukleosidtriphosphat | variabel | — | 10 μl |
| ⁷PPᵢPFK-Pr\overline{a}paration | variabel | 20 μl | 10 μl ^{±} × μl |
| Aldolase | 0.09 U/200 μl | 4 μl | 4 μl |
| ⁸Triosephosphat-Isomerase | 1 U/200 μl | 4 μl | 4 μl |
| ⁹Glycerin-3-Phosphat- | 0.136 U/200 μl | 4 μl | 4 μl |
| Dehydrogenase | | | |
| ¹⁰Pyrophosphorylase- | ¹¹variabel | — | 20 μl |
| Pr\overline{a}paration | | | |

Key:
¹Final concentration
²PPᵢPFK test
³Pyrophosphorylase test
⁴Fructose-6-phosphate
⁵Sugar-1-phosphate
⁶Nucleoside triphosphate
⁷PPᵢPFK preparation
⁸Triose phosphate-isomerase
⁹Glycerin-3-phosphate dehydrogenase
¹⁰Pyrophosphorylase preparation
¹¹Variable

The total volume was 200 μL. The preparations were measured in a Titertek photometer molecular device, Munich, by photometry. PPᵢ was used for the start, in the case of the PPᵢPFK test, and a sugar-1-phosphate or the nucleoside triphosphate was used in the case of pyrophosphorylases.

The following formula was used to calculate the activity:

$$\begin{matrix}\text{U/mL} = ( Δ E ( mOD / time ) * sample dilution * measured volume ) / \\ ( 1000 * sample volume * d * {ϵ}_{NADH} * 2 ) \\ = ( {10}^{- 3} Δ E / min * sample dilution * 200 µL ) / ( {10}^{3} * \\ 20 µL * 0.67 cm * 6.3 ( 1 * mmol {/}^{- 1} * {cm}^{- 1} ) * 2 ) \\ = Δ E * sample dilution * 0.0012 µmol / mL * min\end{matrix}$$

Use of the NUSSA enzyme test

1) Example see above: Activity measurement of the GDP-mannose pyrophosphorylase

2) Example: Use of NUSSA for screening pyrophoshorylases in two different enzyme sources:

a) Escherichia coli BL21(DE3)pLysSpERJ-1

b) Rice (Oryza sativa L.)

For using the test, the PPIPFK has to be purified. As a simple and easily available enzyme source, potatoes were selected (Solanum tuberosum L.) . The purification was carried out according to the method described by van Schaftingen et al., 1982 in Eur. J. Biochem., Vol. 129, pp. 191-195. The enzyme (PPIPFK) was stored in 25% glycerin at -20° C.

Escherichia Coli BL21(DE3)pLysSpERJ-1 was cultured and broken up as described above. The resulting raw homogenate was centrifuged at 10,000 rpm for 2 min, and at 20° C., then used in the enzyme test (21.02 mg/mL). The rice was broken up according to Elling, 1993, German Patent DE 4,221,595 Cl, at 10,000 rpm, 10 min, 20° C., and it was used as a raw extract (4.26 mg/mL) in the enzyme screening. The substrates tested were:

a) Nucleoside triphosphate: ATP, CTP, GTP, UTP, dTTP, each at 1 mM in the test with glucose-1-phosphate (2.5 mM)

b) Nucleoside triphosphate UTP (1 mM)

Sugar-1-phosphate, each at 2.5 mM in the test

Table VII shows the specific activities of pyrophosphorylases in a microbial and in a eukaryotic enzyme source.

TABLE VII
Specific activities of pyrophosphorylases in E. coli and rice

| ¹α-D-Zucker-1-Phosphate | NTPs | E. coli | Reis |
|---|---|---|---|
| [2.5 mM] | [1 mM] | [mU/mg] | [mU/mg] |
| ³α-D-Glucose-1-Phosphat | + ATP | 2.76 | — |
| | + CTP | 0.75 | — |
| | + GTP | — | — |
| | + UTP | 214.10 | 982.40 |
| | + dTTP | 10.32 | 5.98 |
| ⁴α-D-Glucosamin-1-P | + UTP | 2.75 | — |
| α-D-GlcNAc-1 | + UTP | 2.02 | 0.27 |
| ⁵α-D-Galactose-1-P | + UTP | 1 | 10.41 |
| α-D-Galactosamin-1-P | + UTP | — | 0.25 |
| α-D-GalNAc-1-P | + UTP | — | 0.18 |
| ⁶α-D-Glucurons\overline{a}ure-1-P | + UTP | 4.72 | 14.27 |
| ⁷α-D-Galacrurons\overline{a}ure-1-P | + UTP | 3.66 | 2.94 |
| α-D-Xylose-1-P | + UTP | — | 0.42 |
| α-D-Manose-2-P | + GTP | 3.41 | 1.51 |

⁸20 μg/ml E. coli BL21(DE3)pLysS-pJER-1-Rohextrakt
im Testansatz, 1.06 μg/ml bis 0.14 mg/ml Oryza sativa-Rohextrakt
im Testansatz. Aktivit\overline{a}tsbestimung per NUSSA
Key:
¹α-D-Sugar-1-phosphates (2.5 mM)
²Rice (mU/mg)
³α-D-Glucose-1-phosphate
⁴α-D-Glucosamine-1-P
⁵α-D-Galactosamine-1-P
⁶α-D-Glucuronic acid-1-P
⁷α-D-Galacturonic acid-1-P
⁸20 μg/mL E. coli BL21(DE3)pLysS-pJER-1 raw extract in the test preparation; 1.06 μg/mL-0.14 mg/mL of Oryza sativa raw extract in the test preparation. Activity determination by NUSSA

For the analysis of the synthesis of the GDP-mannose, a capillary electrophoresis apparatus (Beckman company) was used. The method used was capillary zone electrophoresis with a borate buffer system. For this purpose, 40 mL of 0.4 mM boric acid and 20 mL of 0.1 mM sodium borate were mixed and the volume was brought up with 140 mL H₂O. The pH was approximately 8.3 for this mixture ratio. The voltage that was preselected was 25 kV. The current established was approximately 35-37μÅ.

To be able to determine the concentrations from the electrophoregrams, different concentrations, 0.02-0.4mM, of GDP-mannose and GTP were prepared in 50 mL Tris-HCl, pH 8, with 5 mM MgCl₂, and analyzed by capillary electrophoresis. By plotting the area versus the theoretically used concentration (mM), a straight line was obtained, so that linear regression can be applied:

| GTP | y = b * x + a with | a: | 0.0534 |
|---|---|---|---|
| | | b: | 3.5314 |
| | | r: | 0.991 |
| | Total [mean]square error: | | 0.4024 * 10⁻² |
| GDP-mannose | y = b * x + a with | a: | 0.0552 |
| | | b: | 3.4742 |
| | | r: | 0.994 |

| | |
|---|---|
| Total square error: | 0 -7178 * 10⁻³ |

Isolation of phosphomannomutase from E. coli BL21(DE3)

Culturing of E. coli BL21 and breakup of the cells: 37° C. in a shaker at 120 rpm, as for GDP-mannose phrophosphorylase

The raw extract obtained was loaded on an anion exchanger:

Q-Sepharose FF:

7 mL of Q-Sepharose FF were loaded with 122 mL of raw extract (with a 14 mg/mL protein content). A linearly increasing gradient (50 mM Tris-HCl, pH 8, 0.600 mM KCl) was prepared, then the protein was eluted between 280 and 460 mM KCl. The active fractions were combined, then the protein was precipitated with 3M (NH₄) ₂SO4. After centrifugation (15 min, 10,000 rpm, 4° C.) the pellet was dissolved in 5 mL Tris-HCl, pH 8. The enzyme could be obtained with a purification factor of 2.4 and a yield of 78%.

TABLE VIII
Partial purification of the phosphomannomutase

| | Gesamt- | Gesamt- | spezifische | | Reini- | |
|---|---|---|---|---|---|---|
| | protein | Aktivit\overline{a}t | Aktivit\overline{a}t | Volumen | gungs- | Ausbeute |
| | [mg]¹ | [U]² | [U/mgl]³ | [ml]⁴ | faktor⁵ | [%]⁶ |
| Rohextrakt⁷ | 1703.1 | 187.9 | 0.11 | 112 | 1 | 100 |
| Q-Sepharose⁸ | 554.2 | 146.5 | 0.26 | 240 | 2.4 | 78 |

Key:
¹Total protein (mg)
²Total activity (U)
³Specific activity (U/mg · L)
⁴Volume (mL)
⁵Purification factor
⁶Yield (%)
⁷Raw extract
⁸Q-Sepharose

Ezymatic synthesis of GDP-α-D-mannose starting with mannose

The enzymatic synthesis of GDP-mannose (FIG. 14) is carried out starting with mannose via the hexokinase-catalyzed phosphorylation at C6, isomerization of mannose-6-phosphate to of mannose-1-phosphate by the phosphomannomutase, and the conversion of mannose-1-phosphate with GTP to GDP-mannose with the GDP-mannose pyrophosphorylase.

The ATP used is recycled, by the conversion of the ADP produced during the hexokinase reaction, with phosphoenol- pyruvate and catalysis by the pyruvate kinase, to form ATP and pyruvate (Wong et al., 1995, Angew. Chem., Vol. 107, pp. 569-593) (FIG. 15).

The synthesis on a larger scale was carried out in the “repetitive batch” procedure. The total volume of the synthetic preparation was 80 mL. The following table shows the composition of said synthetic preparation:

| | ¹Eingesetzte Menge | ²Endkonzentration | |
|---|---|---|---|
| GTP | 253 mg | 5 | mM |
| Mannose | 72.1 mg | 5 | mM |
| PEP | 225 mg | 7.5 | mM |
| ATP | 88.2 mg | 2 | mM |
| Glc-1, 6-P₂ | 1 mg | 0.25 | mM |
| Hexokinase | | 1 | U/ml |
| ³Pyruvat-Kinase | | 40 | U/ml |
| PMM | | 1 | U/ml |
| GDPM-PP | | 1 | U/ml |
| PPase | | 2 | U/ml |
| Tris-HCl, pH 8 | | 50 | mM |
| MgCl₂ | | 10 | mM |
| KCl | | 10 | mM |

Key:
¹Quantity used
²Final concentration
³Pyruvate kinase
⁴PMM: Phosphomannomutase; PPase: Pyrophosphatase; GPDM-PP: GDP-α-D-mannose pyrophosphorylase

After 24 h, the preparation was reduced using an ultrafiltration module with a YM 10 membrane (cutoff 10 kD) from the Amicon company (Witten) to 5 mL; the volume was brought up to 50 mL with 50 mM Tris-HCl, pH 8, 10 mM KC1, and 10 mM MgCl₂. It was reduced again and the volume was again brought up, followed by a renewed reduction to 5 mL. The protein-containing retentate was reacted with a new synthetic preparation with substrate solution (75 mL), and again incubated for 24 h. This protocol was then repeated one more time.

The filtrates were reacted with alkaline phosphatase (1 U/mL) and incubated for 24 h in order to dephosphorylate nucleoside mono-, di-, and triphosphates or sugar phosphates such as mannose-6- or 1-phosphate. The activated sugars are not attacked by the phosphatase.

All in all, 253 mg (0.4 mmol) of GTP were reacted three times with 216.3 mg of mannose. A determination of the yields after 24 h, for each reaction, produced:

| | | Yield |
|---|---|---|
| Preparation 1: | 4.4 mM GDP-mannose | 88% |
| Preparation 2: | 4.8 mM GDP-mannose | 96% |
| Preparation 3: | 2.8 mM GDP-mannose | 56% |
| Mean yield in 72 h: | | 80% |

This corresponds to 581 mg of GDP mannose, with respect to free acid (605.3 g/mol).

After the incubation with alkaline phosphatase, the preparations were ultrafiltered and purified, then loaded on an anion exchanger Dowex® 1×2 Cl⁻, Serva.

The GDP-mannose was eluted using a linear gradient between 0 and 0.5M LiCl (500 mL) with 1M LiCl. The GDP-mannose containing solution (900 mL with 0.92 mM GDP-mannose) was reduced using a rotatory evaporator. This fraction was subjected to gel filtration using Sephadex G-10, then the GDP-mannose-containing fractions were lyophilized. The lyophilizate was dissolved in a small amount of water, then reacted with ice cold acetone. The precipitated GDP-mannose was removed by filtration, dissolved in water, lyophilized, and analyzed by capillary electrophoresis with a comparison of the areas to a standard curve. FIG. 16 shows the electrophoregram of the nondiluted sample (1 mg of the lyophilizate/mL water).

A total of 199 mg of GDP-mannose in 500 mg of lyophilizate was obtained.

1. Method for the preparation of GDP-mannose, in which the gene expression of phosphomannomutase or GDP-mannose pyrophosphorylase (GDP-Man-PP) in a microorganism is increased.

2. Method according to claim 1, characterized in that the gene expression of phosphomannomutase (rfbK) or GDP-Man-PP (rfbM) is increased by invreasing the number of copies of the rfbK or rfbM genes.

3. Method according to claim 2, characterized in that, to increase the number of the copies of the rfbK or rfbM genes, a gene construct is invorporated.

4. Method according to claim 3, characterized in that a microorganism is transformed with the gene construct that contains the rfbK or rfbM gene.

5. Method according to claim 3, characterized in that an Escherichia coli strain is transformed with the gene construct that contains the rfbK or rfbM gene.

6. Method according to claim 5, characterized in that E. coli BL21 (DE3) is transformed with the gene construct.

7. Method according to one of the preceding claims, characterized in that the genes are isolated from a microorganism.

8. Method according to claim 7, characterized in that the genes are isolated from Salmonella enterica, group B.

9. Method according to one of the preceding claims, characterized in that, after increasing the gene expression, the phosphomannomutase or GDP-Man-PP is isolated.

10. Method according to claim 9, characterized in that, for the isolation of the enzymes, the raw extract of the recombinant strain is loaded on an anionic exchanger.

11. Method according to claim 10, characterized in that, for the isolation of the GDP-Man-PP, the ion exchanger is subjected to a stepwise gradient elution, from whose enzyme-enriched fraction the GDP-Man-PP is obtained by hydrophobic interaction chromatography (HIC) with a linearly decreasing (NH₄)₂SO₄ gradient.

12. Method according to one of the preceding claims, characterized in that the phosphomannomutase formed after the increase in the gene expression is used for the reaction of mannose-6-phosphate to form mannose-1-phosphate.

13. Method according to one of the preceding claims, characterized in that the GDP-Man-PP formed after the increase in the gene expression is used with GTP for converting mannose-1-phosphate to GDP-mannose.

14. Mannose- or mannose-derivative-specific GDP-Man-PP, which can be isolated from recombinant cells, having a specific activity≧2 U/mg.

15. Phosphomannomutase, which can be obtained by the method according to one of claims 1 to 10.

16. Transformed cell, containing phosphomannomutase or GDP-Man-PP in overexpressed form.

17. Transformed cell according to claim 16, characterized in that it is Escherichia coli.

18. Transformed cell according to claim 17, characterized in that it is Escherichia coli BL21(DE3).

19. Photometric test in which the pyrophosphate obtained by means of a pyrophosphate releasing enzyme is converted by means of a pyrophosphate-dependent phosphofructokinase, an aldolase, a triose phosphate isomerase, and a glycerin-3-phosphate dehydrogenase, with the reduction that occurs as a result of the dehydrogenase being photometrically determined.

20. Photometric test according to claim 19 for the determination of pyrophosphate-releasing nucleotidyl transferases.

21. Photometric test according to claim 20 for the determination of the GDP-mannose pyrophosphorylase.
